(19) United States
(12) Patent Application Publication
(10) Pub. No.: US 2025/0281518 A1
(43) Pub. Date: Sep. 11, 2025

(54) METHODS AND AGENTS FOR PREVENTING SKELETAL AGING, OSTEOPOROSIS AND OBESITY

(71) Applicant: The Regents of the University of California, Oakland, CA (US)

(72) Inventor: Cun-Yu WANG, Los Angeles, CA (US)

(73) Assignee: The Regents of the University of California, Oakland, CA (US)

(21) Appl. No.: 18/859,754

(22) PCT Filed: Apr. 26, 2023
(86) PCT No.: PCT/US2023/020050

§ 371 Date: Oct. 24, 2024

Related U.S. Application Data
(60) Provisional application No. 63/335,332, filed on Apr. 27, 2022.

(51) Int. Cl. A61K 31/706; A61K 31/19; A61P 19/10

(52) U.S. Cl. CPC A61K 31/706; A61K 31/19; A61P 19/10

(57) ABSTRACT

Methods are provided for treating skeletal aging, osteoporosis and obesity in a subject by therapeutically effective amounts of a combination of alpha-ketoglutarate (α-KG) and nicotinamide mononucleotide (NMN). The therapeutically effective amounts are synergistic in treating skeletal aging, osteoporosis or obesity. Compositions containing both alpha-ketoglutarate (α-KG) and nicotinamide mononucleotide (NMN) are described.

### CROSS-REFERENCE TO RELATED APPLICATIONS

This application claims priority to U.S. Provisional Patent Application Ser. No. 63/335,332, filed Apr. 27, 2022, the contents of which are incorporated herein by reference in its entirety.

### GOVERNMENT INTEREST STATEMENT

This invention was made with government support under Grant Number DE028260, awarded by the National Institutes of Health. The government has certain rights in the invention.

### BACKGROUND

Aging causes significant changes to the skeleton, characterized by a decrease in trabecular bone and an increase in marrow adiposity. Age-related bone loss is a critical risk factor for osteoporosis that affects millions of patients worldwide. Osteoporosis is the most common metabolic bone disease and a leading cause of morbidity and mortality in expanding aging population. In skeletal aging, osteoporosis and obesity, there is an inverse relationship between bone mass and marrow adiposity. Bone marrow adipose tissue (MAT) accumulation is increased at the expense of bone formation. Moreover, increased MAT accumulation and lower bone density are also hallmark symptoms of patients with radiation and chemotherapy, or having type 1 diabetes. Obesity is another major health care concern with profound effects on these sequelae and those of other organ systems. Therefore, it is important to understand the underlying mechanisms which control the switch of bone and fat in bone marrow.

Bone marrow mesenchymal stem/stromal cells (MSCs) are believed to be the common progenitors for both osteoblasts and adipocytes in bone, but commitments to these two lineages are mutually exclusive. Aging reduces the bone marrow MSC pool and its self-renewal, and impairs their fate commitment and differentiation. Over the past decade, several transcription factors or signaling pathways have been identified in association with MSC fate commitment and differentiation. For example, the master osteogenic factor RUNX2 was found to be a critical factor for MSC fate determination. Induction of RUNX2 inhibits adipogenesis while promoting osteogenesis.

Epigenetic mechanisms further play a critical role in aging, stem cell identity and fate. Stem cell exhaustion and epigenetic alterations are two of the hallmarks of aging.

Skeletal aging, obesity and osteoporosis are major drains on the health care system especially in the older population and as this population proportionately increases. Methods for effectively and economically addressing these conditions and diseases are highly desirable.

### SUMMARY

In one aspect, a method is provided for treating skeletal aging, osteoporosis or obesity in a subject comprising administering to the subject therapeutically effective amounts of alpha-ketoglutarate (α-KG) and nicotinamide mononucleotide (NMN). In some embodiments, the therapeutically effective amounts of α-KG and NMN are synergistic in treating skeletal aging, osteoporosis or obesity.

In some embodiments, the administering of a therapeutic combination of α-KG and NMN promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof. In some embodiments, the administration decreases body weight during aging. In some embodiments, the subject is overweight, obese, has a high BMI, has type 1 diabetes mellitus, has undergone radiation therapy or has undergone chemotherapy. In some embodiments, the subject has a Western diet.

In some embodiments, the α-KG and NMN are administered in the same dosage form. In some embodiments, the α-KG and NMN are administered in separate dosage forms. In some embodiments, the α-KG and NMN are each administered daily. In some embodiments, the α-KG and NMN are each administered more often than once daily. In some embodiments, the α-KG and NMN are each administered every other day. In some embodiments, the α-KG and NMN are each administered on alternate days. In some embodiments, the α-KG and the NMN are each administered orally. In some embodiments, the α-KG and the NMN are administered chronically. In some embodiments, the α-KG and NMN are administered separately, in an overlapping schedule.

In some embodiments, the daily dose of α-KG is about 100-1000 mg/day and the daily dose of NMN is about 100-1000 mg/day.

In some embodiments, an active analogue of either α-KG, NMN or both are administered. In some embodiments, the active analogue of NMN is nicotinamide riboside.

In one aspect, a composition is provided comprising therapeutically effective amounts of α-KG) and NMN, for use in treating skeletal aging, osteoporosis or obesity in a subject. In some embodiments, the therapeutically effective amounts of alpha-ketoglutarate (α-KG) and nicotinamide mononucleotide (NMN) in the composition are synergistic in treating skeletal aging, osteoporosis or obesity.

In one embodiment, administration of the composition promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof. In one embodiment, administration of the composition decreases body weight during aging. In some embodiments, the subject is overweight, obese, has a high BMI, has type 1 diabetes mellitus, has undergone radiation therapy or has undergone chemotherapy. In some embodiments, the subject has a Western diet.

In some embodiments, the composition is provided for daily administration. In some embodiments, the composition is provided for oral administration. In some embodiments, the composition comprises about 100-1000 mg α-KG and about 100-1000 mg NMN. In some embodiments, the composition comprises an active analogue of either α-KG, NMN or both. In some embodiments, the active analogue of NMN is nicotinamide riboside.

### BRIEF DESCRIPTION OF THE DRAWINGS

The subject matter disclosed herein is particularly pointed out and distinctly claimed in the concluding portion of the specification. The disclosure, however, both as to organization and method of operation, together with objects, features, and advantages thereof, may best be understood by reference to the following detailed description when read with the accompanying drawings in which:

FIG. 1 shows that alpha-ketoglutarate (α-KG) and nicotinamide mononucleotide (NMN) synergistically reduced mouse body weight gains induced by ovariectomy (OVX). A. α-KG and NMN did not affect mouse daily food consumption. B. aα-KG and NMN did not affect mouse daily water consumption. C and D. α-KG and NMN collaboratively reduced mouse body weight gain upon OVX. E. α-KG and NMN collaboratively reduced fact mass upon OVX. In all figures, group labels with 100 refer to 100 mg/kg/day, labels with 200 refer to 200 mg/kg/day, and labels with 500 refer to 500 mg/kg/day, for compounds individually or in combination.

FIG. 2 shows that α-KG and NMN synergistically reduce mouse liver lipids upon OVX. A. α-KG and NMN reduced mouse liver weight gain induced by OVX. B. Histology showing that α-KG and NMN collaboratively reduced mouse liver fat deposits upon OVX. C. α-KG and NMN collaboratively reduced liver lipids gains upon OVX in mice. *p<0.05; **p<0.01; ***p<0.001.

FIG. 3 shows that α-KG and NMN synergistically reduce mouse inguinal white adipose tissue (iWAT) upon OVX. A. α-KG and NMN reduced mouse iWAT weight gain induced by OVX. B. α-KG and NMN collaboratively reduced mouse iWAT lipid droplets upon OVX in mice. C. Histology showing that α-KG and NMN collaboratively reduced mouse iWAT fat cell size OVX *p<0.05; **p<0.01; ***p<0.001.

FIG. 4 shows that α-KG and NMN synergistically reduce mouse peri-gonadal white adipose tissue (pgWAT) upon OVX. A. α-KG and NMN reduced mouse pgWAT weight gain induced by OVX. B. α-KG and NMN collaboratively reduced mouse pgWAT lipid droplets upon OVX in mice. C. Histology showing that α-KG and NMN collaboratively reduced mouse pgWAT fat cell size OVX *p<0.05; **p<0.01; ***p<0.001.

FIG. 5 shows that α-KG and NMN synergistically prevent trabecular bone loss upon OVX. A. Micro-CT (μCT) analysis of trabecular bone. B. α-KG and NMN collaboratively prevented bone mineral density (BMD) loss upon OVX in mice. C. α-KG and NMN collaboratively prevented bone volume/trabecular volume (BV/TV) loss upon OVX in mice. C and D. μCT analysis showed that α-KG and NMN collaboratively prevented regulated marrow adipose tissue (rMAT) accumulation in bone marrow upon OVX. *p<0.05; **p<0.01; ***p<0.001.

FIG. 6 shows α-KG and NMN synergistically reduced mouse body weight gains induced by high fat diet (HFD). A. Mouse body weight before treatment. B. Mouse body weights after treatment. C. The percentage of mouse body weight gains after treatment. D. α-KG and NMN collaboratively reduced mouse fat mass upon HFD. *<0.05; **<0.01; ***p<0.001.

FIG. 7 shows α-KG and NMN synergistically reduce mouse liver lipid accumulation induced by HFD. A. α-KG and NMN collaboratively reduced mouse liver weight gains induced by HFD. B. Histology showing that α-KG and NMN collaboratively reduced mouse liver fat deposits induced by HFD. *p<0.05; **p<0.01; ***p<0.001.

FIG. 8 shows α-KG and NMN synergistically reduce mouse pgWAT induced by HFD. A. α-KG and NMN reduced mouse pgWAT weight gain induced by HFD. B. Histology showing that α-KG and NMN collaboratively reduced mouse pgWAT fat cell sizes induced by HFD. *p<0.05; **p<0.01; ***p<0.001.

FIG. 9 shows that α-KG and NMN synergistically prevent trabecular bone loss upon OVX. A. mCT analysis of trabecular bone showing that α-KG and NMN collaboratively prevented HFD-induced bone mineral density (BMD) loss in mice. B. α-KG and NMN collaboratively prevented HFD-induced BV/TV loss in mice. *p<0.05; **p<0.01; ***p<0.001.

FIG. 10 shows α-KG and NMN synergistically reduced mouse aging-related body weight gains. A. α-KG and NMN did not affect mouse daily water consumption. B. α-KG and NMN did not affect mouse daily food consumption. C. The percentage of mouse body weight gains after 6-month treatment. D. α-KG and NMN collaboratively reduced mouse fat mass. *<0.05; **<0.01; ***p<0.001;****p<0.0001.

FIG. 11 shows α-KG and NMN synergistically reduce mouse aging-related liver lipid and pgWAT accumulation. A. α-KG and NMN collaboratively reduced mouse aging-related liver weight gains. B. α-KG and NMN reduced mouse aging-related pgWAT weight gain. *p<0.05; **p<0.01; ***p<0.001; ****p<0.0001.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the disclosure embodied herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present disclosure and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

In one aspect, a method is described for treating skeletal aging, osteoporosis or obesity in a subject comprising administering to the subject therapeutically effective amounts of alpha-ketoglutarate (α-KG) and nicotinamide mononucleotide (NMN). In some embodiments, the therapeutically effective amounts of alpha-ketoglutarate (α-KG) and nicotinamide mononucleotide (NMN) are synergistic in treating skeletal aging, osteoporosis or obesity. Synergistic, collaboratively, or synonyms thereof, indicate that the effect of the therapeutic combination is greater than the effect obtained by adding the effect obtained from either compound alone at the same dose level. The greater effect may be observed in any one, or in any more than one, of any outcomes or measures of the effect of the combination on one or more sequelae of skeletal aging, osteoporosis or obesity. Such outcomes, as will be described below, include molecular, cellular and metabolic effects as well as effects on food consumption, body weight, organ weights, organ composition, fat content, composition or distribution, by way of non-limiting examples.

In some embodiments, the therapeutic combination of doses of α-KG and NMN is effective in one or more outcomes where either α-KG or NMN alone is ineffective at one or more outcomes. In one non-limiting example, in OVX mice, the combination of 100 mg/kg/day α-KG and 100 mg/kg/day NMN is effective in suppressing weight gain, where either 100 mg/kg/day α-KG alone or 100 mg/kg/day NMN alone is ineffective in suppressing weight gain. This, in some embodiments, a combination of α-KG and NMN is effective at one or more of: promoting osteogenic differentiation, increasing in trabecular bone, inhibiting adipogenic differentiation of bone marrow mesenchymal cells, inhibiting adipogenic differentiation of bone marrow stromal cells, increasing bone mass, reducing bone marrow adiposity, preventing bone loss, increasing the ratio of osteogenesis to adipogenesis in bone marrow mesenchymal cells, or any combination thereof, whereas either α-KG or NMN alone is ineffective.

In some embodiments, the combination of α-KG and NMN has a greater effect on promoting osteogenic differentiation, increasing in trabecular bone, inhibiting adipogenic differentiation of bone marrow mesenchymal cells, inhibiting adipogenic differentiation of bone marrow stromal cells, increasing bone mass, reducing bone marrow adiposity, preventing bone loss, increasing the ratio of osteogenesis to adipogenesis in bone marrow mesenchymal cells, or any combination thereof, whereas either α-KG or NMN alone has a less effect thereon. In one embodiment the effect of the combination of α-KG and NMN on any of the outcomes is greater than the additive effect of α-KG and of NMN when each is administered alone. In one embodiment, the greater effect is significantly greater than the additive effect. In one embodiment, the greater effect is at least 20% greater than the additive effect. In one embodiment, the greater effect is at least 40% greater than the additive effect. In one embodiment, the greater effect is at least 50% greater than the additive effect. In one embodiment, the greater effect is at least 60% greater than the additive effect. In one embodiment, the greater effect is at least 80% greater than the additive effect. In one embodiment, the greater effect is at least 100% greater (i.e., at least twice) than the additive effect. In one embodiment, the greater effect is more than 100% greater than the additive effect.

In some embodiments, the effect of the therapeutic combination is equivalent that that obtained from one of the compounds at a higher dose level, such that a lower dose of one of the compounds in combination with the other results in an extent of effect obtained by one compound at a dose of at least 50%, at least 100%, at least 200%, at least 300%, at least 400% or at least 500% higher. By way of non-limiting example, the combination of 100 mg/kg/day α-KG and 100 mg/kg/day NMN results in the reduced body weight gain of OVX animals that 500 mg/kg/day α-KG alone or 500 mg/kg/day NMN alone produce.

Alpha ketoglutarate (α-KG) is a crucial intermediate in the tricarboxylic acid (TCA) cycle, locating between succinyl-CoA and isocitrate. As a key point of anaplerotic reaction, α-KG regulates ATP production and reduces equivalent (NAD+/NADH) generation in TCA cycle, therefore influencing reactive oxygen species (ROS) levels and immune system homeostasis. Moreover, α-KG is an important source of glutamine and glutamate that are required for synthesis of both amino acid and collagen. α-KG, also called α-ketoglutaric acid, 2-ketoglutaric acid, 2-oxoglutaric acid, among other chemical names, has CAS number 328-50-7, a chemical formula C₅H₆O₅ and a molar mass of 146.11 g/mol. α-KG is available as a nutritional supplement in tablets or capsules of unit doses of, for example, 100 mg, 300 mg, 500 mg and 1600 mg.

Nicotinamide mononucleotide (NMN), also known chemically as 3-carbamoyl-1-[5-O-(hydroxyphosphinato)-β-D-ribofuranosyl]pyridinium, and under the synonyms nicotinamide ribonucleoside 5′-phosphate, nicotinamide D-ribonucleotide, β-nicotinamide ribose monophosphate and nicotinamide nucleotide, has a CAS number 1094-61-7, a chemical formula C₁₁H₁₅N₂O₈P, and a molar mass of 334.221 g/mol. NMN is a nucleotide derived from ribose and nicotinamide. Like nicotinamide riboside, NMN is a derivative of niacin, and humans have enzymes that can use NMN to generate nicotinamide adenine dinucleotide (NADH). NMN is available as a nutritional supplement in tablets or capsules of, for example, 125 mg, 250 mg, 300 mg and 500 mg for oral or sublingual use.

Aging is characterized by the chronical and gradual deterioration of the functional capacities at the cellular, tissue, and organismal level, leading the individual more prone to various diseases, among which osteoporosis is the most representative illness of skeleton aging. Osteoporosis is the systemic loss of bone mass and the significant degradation in mechanical properties, which subsequently lead to a higher risk of bone fracture. As aging population is dramatically rising worldwide, the incidence of osteoporosis among aging populations is significantly increasing as well, which has brought a huge burden on public health. The compositions described here are useful for the treatment of aging including osteoporosis and skeletal aging.

Aging-associated diseases create a heavy economic and social burden for our society. Skeletal aging frequently leads to significant morbidity and mortality in older populations, including a gradual erosion in quality of life. Skeletal aging is characterized by a decrease in the adult stem cell pool, self-renewal, trabecular bone formation, and an increase in marrow adipose tissue accumulation. The compositions described here are useful for the treatment of skeletal aging.

Osteoporosis, and in particular age-related osteoporosis, is characterized by the deterioration in bone volume and strength, partly due to the dysfunction of mesenchymal stem cells (MSCs) during aging. The compositions described here are useful for the treatment of osteoporosis.

Age-related disorders such as tumor, metabolic disease, memory deterioration, and immunologic degeneration, are associated with declined regenerative capacity in rapidly dividing stem cells. The compositions embodied herein are useful for the treatment of age-related disorders.

Obesity is a major risk factor for many adverse health consequences including longevity and osteoporosis. Obesity affects bone metabolism and has a detrimental effect on bone quality. Obesity in combination with aging increases the risk. The compositions embodied herein are useful for the treatment of obesity.

In some embodiments, the administering of a therapeutic combination of α-KG and NMN promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof. In some embodiments, the administration decreases body weight during aging. In some embodiments, the subject is overweight, obese, has a high BMI, has type 1 diabetes mellitus, has undergone radiation therapy or has undergone chemotherapy. In some embodiments, the subject has a Western diet.

As noted above, a subject may be identified as a candidate for therapy by a composition described here upon diagnosis of skeletal aging, osteoporosis or obesity, or is at risk for development. Skeletal aging, osteoporosis or obesity may be identified and monitored in a subject by measuring any of the foregoing effects, such as but not limited assessing osteogenic differentiation, identifying a decrease in trabecular bone, identifying adipogenic differentiation of bone marrow mesenchymal cells, identifying adipogenic differentiation of bone marrow stromal cells, decease in bone mass, increase in bone marrow adiposity, increase in bone loss, decrease in the ratio of osteogenesis to adipogenesis in bone marrow mesenchymal cells, or any combination thereof. In some embodiments, an increase in body weight during aging is an indication for treatment as described herein. In some embodiments, the subject is overweight, obese, has a high BMI, has type 1 diabetes mellitus, has undergone radiation therapy or has undergone chemotherapy. In some embodiments, the subject has a Western diet.

In some embodiments, the α-KG and NMN are administered in the same dosage form. In some embodiments, the dosage form may be a tablet or capsule. In some embodiments, the α-KG and NMN are administered in separate dosage forms. In some embodiments, the dosage forms of each of α-KG and NMN may independently be a tablet or capsule. In some embodiments the oral dosage form may be a liquid or syrup. In some embodiments where oral administration is not facile or possible, an intravenous, subcutaneous or other parenteral dosage form may be provided. In one embodiment, the combination or individual compounds may be administered by inhalation. In some embodiments, the α-KG and NMN are administered in different dosage forms. In some embodiments, the α-KG and NMN are administered by different routes of administration. In some embodiments, the α-KG and NMN are administered at different times of the day.

In some embodiments, the α-KG and NMN are each administered daily. In some embodiments, the α-KG and NMN are each administered more often than once daily. In some embodiments, the α-KG and NMN are each administered every other day. In some embodiments, the α-KG and NMN are each administered on alternate days. In some embodiments, the α-KG and NMN are each administered twice a week or weekly.

In some embodiments, the α-KG and the NMN are each administered orally. In some embodiments, the α-KG and NMN may be administered by other routes such as but not limited to intravenously, subcutaneously and intraperitoneally. A suppository formulation is also embodied herein.

In some embodiments, the α-KG and the NMN are administered chronically. In some embodiments, the combination is administered from the time symptoms of skeletal aging, osteoporosis or obesity appear or are diagnosed, for the rest of the subject's life. In one embodiment, the combination is administered from the time symptoms of skeletal aging, osteoporosis or obesity appear or are diagnosed until the skeletal aging, osteoporosis or obesity stop progressing or become stabilized. In one embodiment, once the symptoms of skeletal aging, osteoporosis or obesity have become reduced or stabilized, a maintenance dose of the combination is administered once a week or once a month. In some embodiments, the combination is administered to prevent the development of skeletal aging, osteoporosis or obesity in a subject with a predisposition thereto, or at an age where the prevalence of skeletal aging, osteoporosis or obesity are elevated.

As will be seen in the examples below, synergy was observed in animal models in which 100 mg/kg/day α-KG and 100 mg/kg/day NMN were administered. The human equivalent doses are about 8.13 mg/kg/day each of α-KG and NMN. In a 60 kg subject, the effective daily dose would be 488 mg/day. One of skill in the art would readily determine in subjects the minimal and optimal efficacious dose levels of α-KG and NMN that provide the desired effect and provide synergy compared to dose levels of α-KG or NMN separately. Such dose levels will be based on oral bioavailability, distribution, metabolism excretion and other factors for each compound in humans. In some embodiments, the dose of α-KG for an average weight human subject is about 10-1000 mg/day and the dose of NMN is about 10-1000 mg/day. In some embodiments, the dose of α-KG for an average weight human subject is about 10-500 mg/day and the dose of NMN is about 10-500 mg/day. In some embodiments, the dose of α-KG for an average weight human subject is about 100-500 mg/day and the dose of NMN is about 100-500 mg/day. In one embodiment, a daily dose comprises 50 mg of α-KG and 50 mg of NMN. In one embodiment, a daily dose comprises 100 mg of α-KG and 100 mg of NMN. In one embodiment a daily dose comprises 300 mg of α-KG and 500 mg of NMN.

In some embodiments, an active analogue of either α-KG, NMN or both are administered. In some embodiments, the active analogue of NMN is nicotinamide riboside.

In one aspect, a composition is provided comprising therapeutically effective amounts of alpha-ketoglutarate (α-KG) and nicotinamide mononucleotide (NMN), for use in treating skeletal aging, osteoporosis or obesity in a subject. In some embodiments, the therapeutically effective amounts of α-KG and NMN in the composition are synergistic in treating skeletal aging, osteoporosis or obesity.

In one embodiment, administration of the composition promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof. In one embodiment, administration of the composition decreases body weight during aging. In some embodiments, the subject is overweight, obese, has a high BMI, has type 1 diabetes mellitus, has undergone radiation therapy or has undergone chemotherapy. In some embodiments, the subject has a Western diet.

In some embodiments, the composition is provided for daily administration. In some embodiments, the composition is provided for oral administration. In some embodiments, the composition comprises for an average weight human subject about 10-500 mg/kg/day alpha-ketoglutarate (α-KG) and about 10-500 mg/day nicotinamide mononucleotide (NMN). In one embodiment, a daily dose comprises a composition of 50 mg of α-KG and 50 mg of NMN. In one embodiment, a daily dose comprises a composition of 100 mg of α-KG and 100 mg of NMN. In one embodiment a daily dose comprises a composition of 300 mg of α-KG and 500 mg of NMN.

In some embodiments the dosage form is a capsule. In some embodiments, the dosage form is a tablet. In some embodiments the capsule or tablet is chewable or for swallowing. In some embodiments the tablet is for sublingual use. In some embodiments the composition is in the form or a suppository. In some embodiments the dosage form is a liquid or syrup. In some embodiments, the dosage form is an intravenous solution, a subcutaneous solution or an intraperitoneal solution.

Provided herein are pharmaceutical compositions comprising a therapeutically effective amount of alpha-ketoglutarate (α-KG) and nicotinamide mononucleotide (NMN). In some embodiments, the pharmaceutical composition further comprises one or more pharmaceutically acceptable carriers, excipients and/or diluents.

“Pharmaceutically acceptable carriers” include any excipient which is nontoxic to the cell or subject being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buffers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions disclosed herein may be formulated in a variety of ways, including for example, solid and liquid dosage forms, such as liquid solutions, dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Pharmaceutical compositions suitable for use include sterile aqueous solutions (where water soluble) or dispersions and sterile powders for the extemporaneous preparation of sterile solutions or dispersions. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of injectable compositions can be brought about by including in the composition an agent which delays absorption, for example, aluminum monostearate and gelatin.

Sterile solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts indicating that the associated compositions are useful for treating a subject suffering skeletal aging, osteoporosis or obesity as described herein.

Effective doses of the compositions disclosed herein, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human, but non-human organisms, including non-human mammals and birds, as well as transgenic organisms, can also be treated. Treatment dosages may be titrated using routine methods known to those of skill in the art to optimize safety and efficacy.

The pharmaceutical compositions disclosed herein may include a “therapeutically effective amount.” A “therapeutically effective amount” refers to an amount effective, at dosages and for periods of time necessary, to achieve the desired therapeutic result. A therapeutically effective amount of a composition of alpha-ketoglutarate (α-KG) and nicotinamide mononucleotide (NMN) may vary according to factors such as the disease state, species, age, sex, and weight of the individual, and the ability of the molecules to elicit a desired response in the individual. A therapeutically effective amount is also one in which any toxic or detrimental effects of the molecule are outweighed by the therapeutically beneficial effects. As noted herein, the doses and dosage forms of the components of the composition are provided in a therapeutic combination. In some embodiments, the doses and dosage forms of the components of the composition are provided in a synergistic combination.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of the active compounds calculated to produce a desired therapeutic effect. In some embodiments, the dosage unit forms disclosed herein are dictated by and directly dependent on the unique characteristics of the active compounds and the particular therapeutic or prophylactic effect to be achieved.

The composition disclosed herein may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, topical, transdermal, oral (for example, in capsules, suspensions or tablets), parenteral (including subcutaneous, intravenous, intramedullary, intraarticular, intramuscular, or intraperitoneal injection), or rectal. Administration to a subject may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

When not otherwise stated, “substantially” means “being largely, but not wholly, that which is specified” (e.g., “substantially pure”).

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In some embodiments, the composition comprises an active analogue of either alpha-ketoglutarate (α-KG), nicotinamide mononucleotide (NMN) or both. In some embodiments, the active analogue of NMN is nicotinamide riboside.

The following examples are presented in order to more fully illustrate the preferred embodiments of the disclosure. It should in no way be construed, however, as limiting the broad scope of the disclosure.

### EXAMPLE 1

### α-KG and NMN Synergistically Prevents Estrogen Deficiency-Induced Osteoporosis and Obesity

Aging-related bone loss and osteoporosis frequently occur in women after menopause. To mimic the pathogenesis of osteoporotic bone loss, we performed ovariectomy (OVX) on female mice to induce estrogen deficiency. OVX accelerates osteoclastic bone resorption while increasing bone formation in a compensatory manner. However, bone formation is unable to compensate for increased bone resorption, resulting in a net bone loss. Interestingly, OVX also induced obesity and body weight gain in female mice. Two separate batches of in vivo experiment were conducted to investigate the effect of α-KG and NMN on bone and adipose metabolism in OVX C57BL/6J female mice. Mice weight on average 20 g at the start of the experiment. Mice were treated with drinking water with α-KG and/or NMN to provide the following daily dose levels: 1) Sham with normal drinking water; 2) OVX with normal drinking water; 3) OVX+100 mg/kg/day α-KG; 4) OVX+100 mg/kg/day NMN; 5) OVX+500 mg/kg/day α-KG; 6) OVX+500 mg/kg/day NMN; 7) OVX+combination 100 mg/kg/day α-KG+100 mg/kg/day NMN; and 8) OVX+combination 200 mg/kg/day α-KG+200 mg/kg/day NMN). Dosing was over two months.

In this and the other experiments described, the concentration of α-KG and/or NMN in drinking water was calculated to provide the specified daily doses, based on daily water consumption and body weights of the animals. For the 100 mg/kg/day dose, OVX animals weighing about 20 grams at the start of the study were given water containing 0.67 mg/mL (4.6 mM) α-KG and/or 0.67 mg/mL NMN (2 mM). For a dose of 200 mg/kg/day, the drinking water contained 1.33 mg/mL (9.2 mM) α-KG and/or 1.33 mg/mL (4 mM). For 500 mg/kg/day, the drinking water contained 3.35 mg/ml (23 mM) α-KG or 3.35 mg/mL (10 mM) NMN. For the mice on high fat diets, the starting animal weights were about 25 grams. In the aging animal study, the mice were about 40 grams at the start of the study.

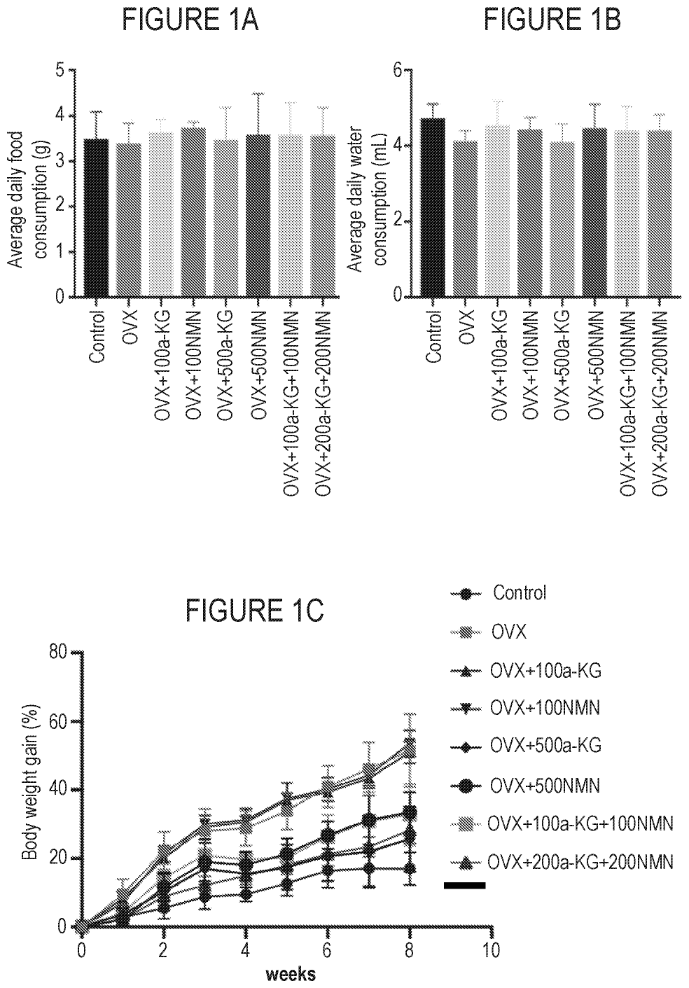

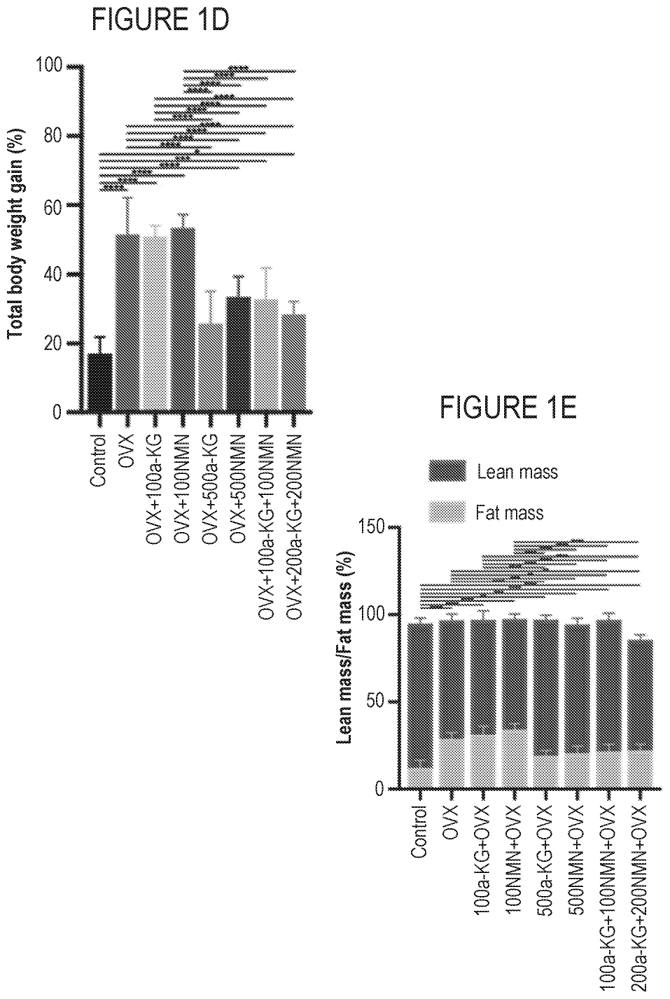

We monitored mouse body weight, food and water consumption on a weekly basis for two months. Nuclear magnetic resonance scanner was used to assess lean mass and fat mass composition. Serum chemistries were analyzed in UCLA DLAM facility. Liver and adipose tissues were harvested for histology and histomorphometric analyses. Femur, tibia and mandible were subjected to micro-CT scanning and reconstruction. For marrow adipose tissue analyses, tibias were decalcified in 1:1 ratio of 2% osmium tetroxide and 5% potassium dichromate solution for 2 days and then μCT scanning was conducted. One-way ANOVA was performed to quantify differences among all the groups. Since α-KG and NMN dissolve well in water and the water solutions are highly stable, mice were fed with regular diet and water containing α-KG or NMN, or the combination. During the entire experiment, no significant differences of food and water intake were detected among all the groups (FIG. 1A and B). In the figures, the dose level is shown for each group as 100 for 100 mg/kg/day, 200 for 200 mg/kg/day and 500 for 500 mg/kg/day, for the α-KG and NMN in combination or individually.

While OVX significantly induced mouse body weight gain compared with sham operation, the treatment with 500 mg/kg/day α-KG or 500 mg/kg/day NMN significantly inhibited mouse body weight gain which α-KG appeared more potent than NMN. Importantly, although treatment with 100 mg/kg/day α-KG or 100 mg/kg/day NMN did not significantly inhibit OVX-induced body weight gain, the combination of 100 mg/kg/day α-KG and 100 mg/kg/day NMN significantly inhibited OVX-induced body weight gain (FIG. 1C and D). Nuclear magnetic resonance analyses also showed similar results for lean mass to fat mass (%) (FIG. 1E).

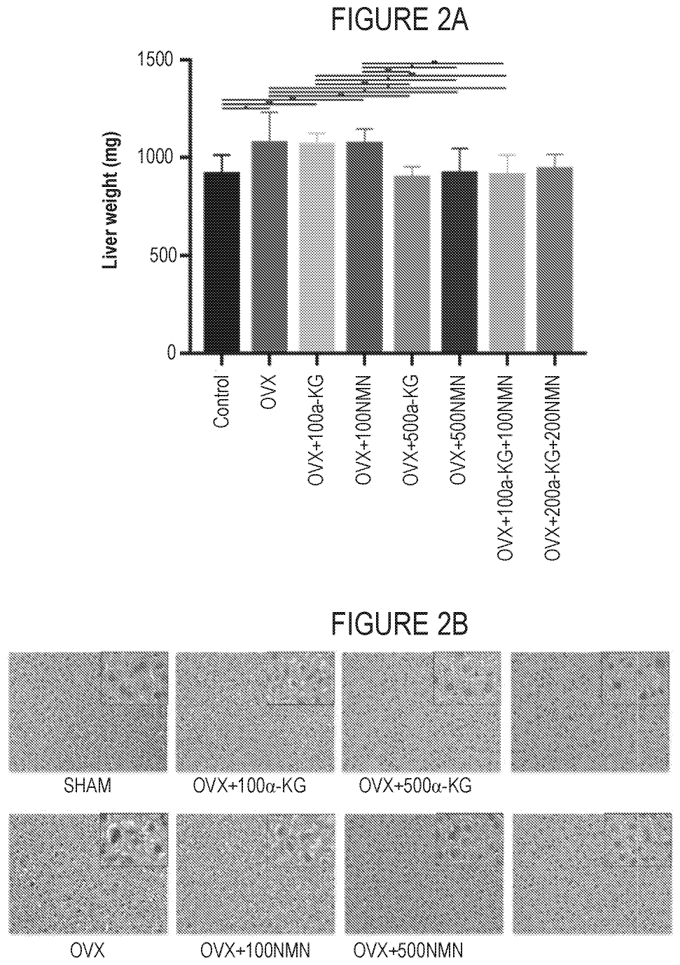

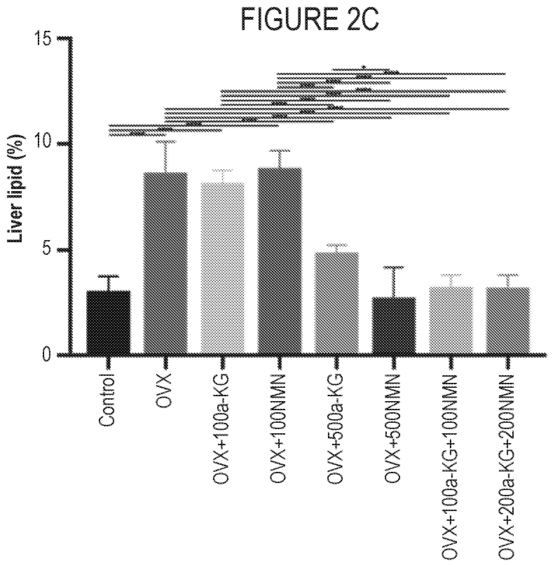

The liver plays a key role in lipid metabolism. The weight and size of the livers were significantly higher, and more lipid was accumulated in the hepatocytes upon OVX. While 100 mg/kg/day α-KG or 100 mg/kg/day NMN alone did not significantly affect the weight and size of liver, the combination of 100 mg/kg/day α-KG and 100 mg/kg/day NMN significantly reduced the gain of the liver weight induced by OVX (FIG. 2A). Similarly, histological analysis also showed that α-KG and NMN also synergistically reduced the content of liver lipids induced by OVX (FIG. 2B and C).

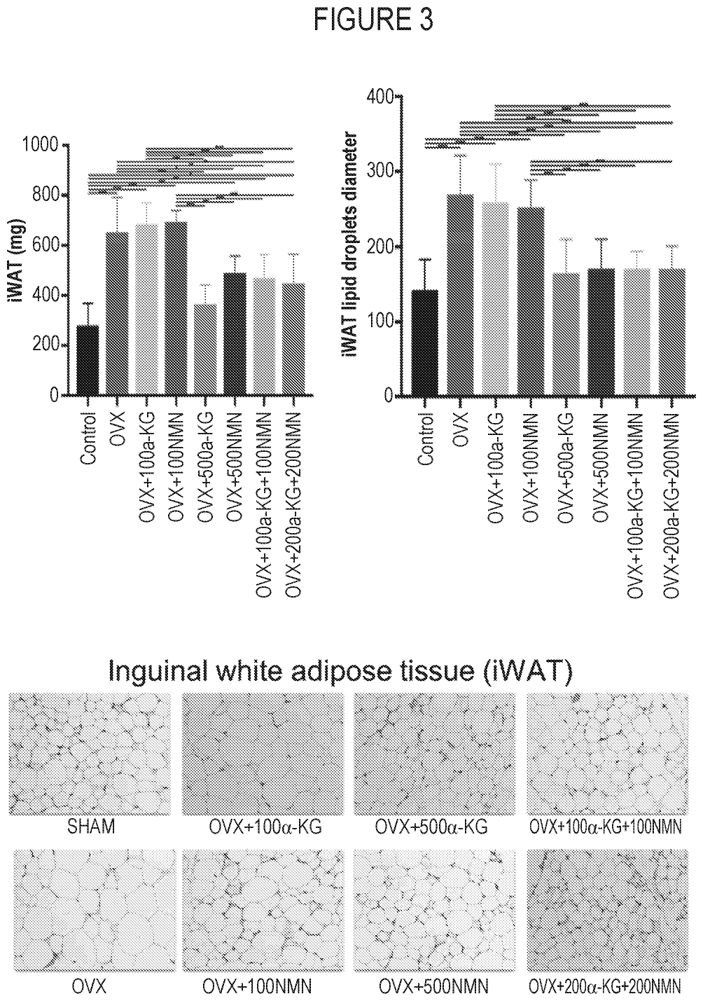

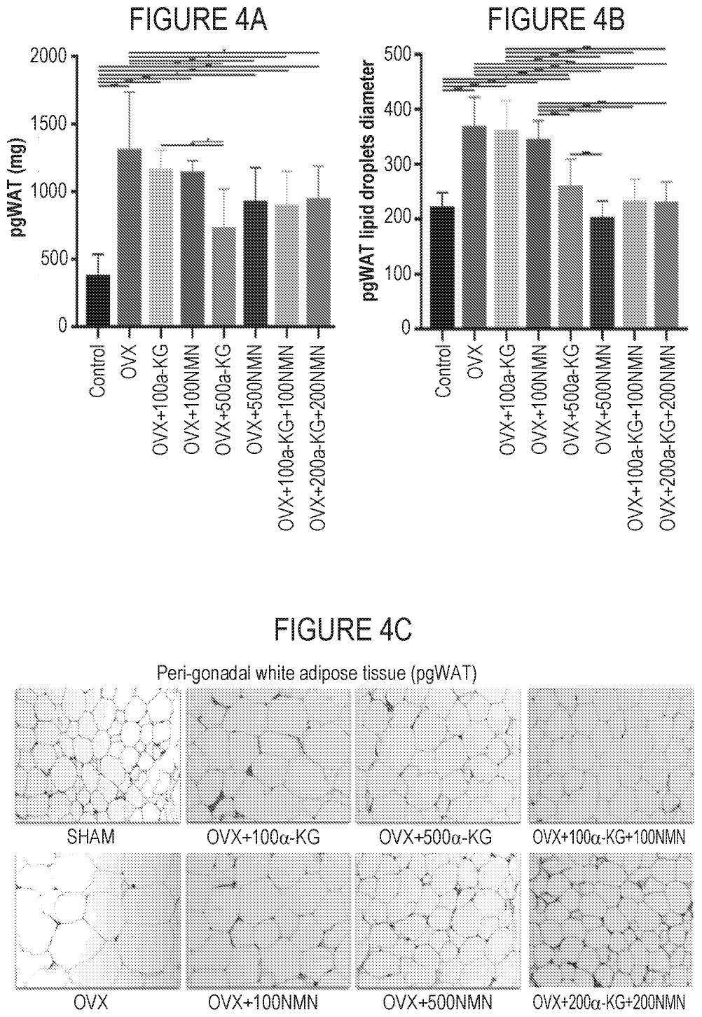

There are two types of adipose tissues found in mammals, brown adipose tissue and white adipose tissue. In rodents, white adipose tissue (WAT) is stored in both visceral and subcutaneous depots and is served as a primary energy reservoir. The most representative and frequently analyzed WAT is inguinal WAT (iWAT) and peri-gonadal WAT (pgWAT). The combination of 100 mg/kg/day α-KG+100 mg/kg/day NMN also synergistically reduced increasing iWAT weights induced by OVX (FIG. 3A). H&E staining and histomorphometric analyses also confirmed that the combination of α-KG and NMN significantly reduced mouse fat cell sizes (FIG. 3B and C). Similarly, we found that the combination of 100 mg/kg/day α-KG+100 mg/kg/day NMN also synergistically reduced increasing pgWAT weight gains induced by OVX (FIG. 4A-C).

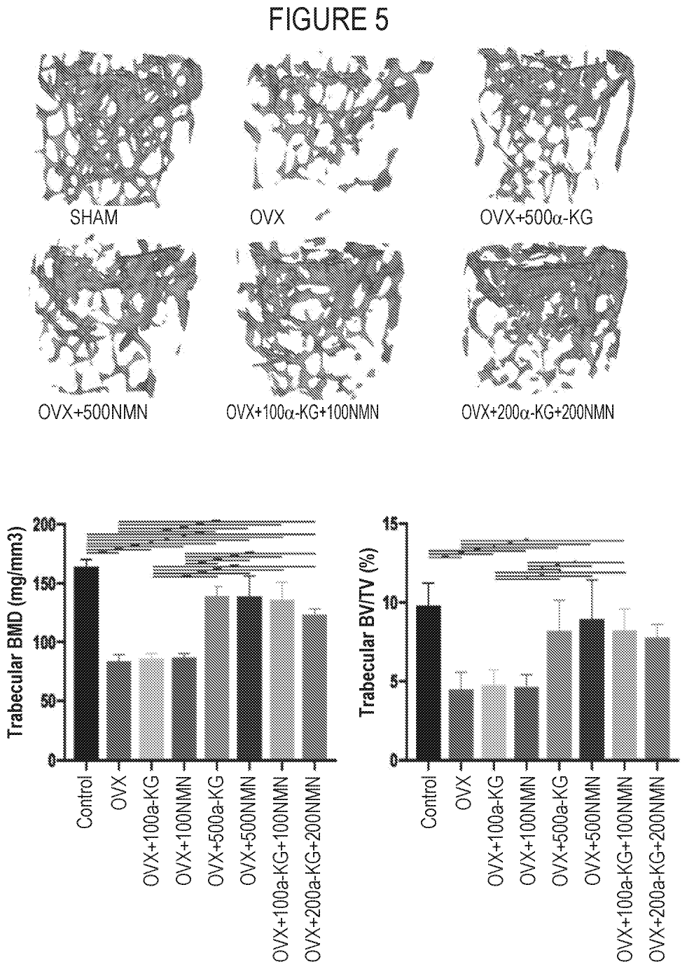

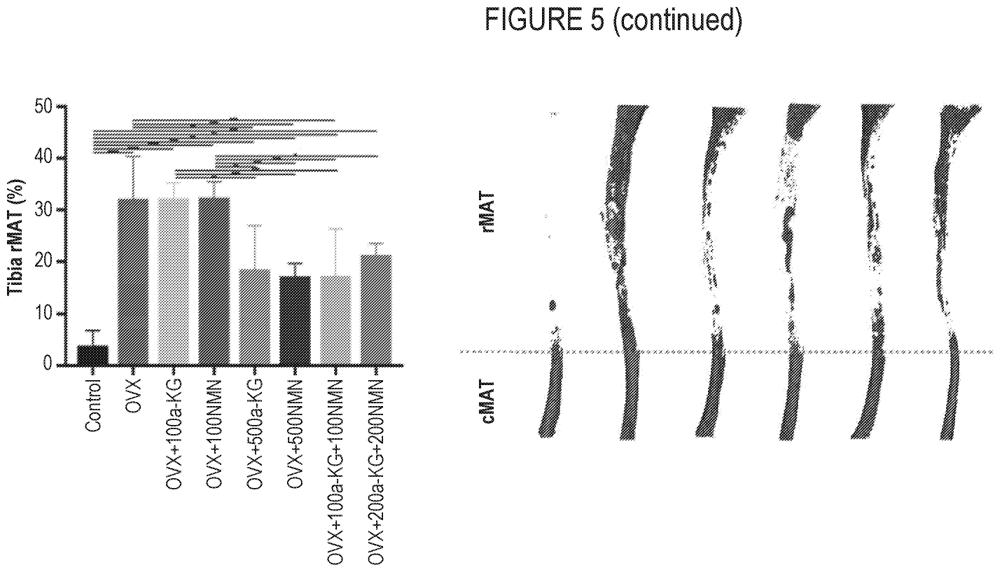

Micro-CT (mCT) analysis of trabecular bones revealed that OVX significantly induced loss of bone mineral density (BMD) and bone volume. The combined treatment of 100 mg/kg/day α-KG and 100 mg/kg/day NMN synergistically prevented decrease in trabecular BMD and BV/TV induced by OVX (FIG. 5A-C). Additionally, we also examined the marrow adipose tissue accumulation (MAT) in mouse bone marrow after OVX. We found that the combined treatment of α-KG and NMN also synergistically reduced regulated MAT (rMAT) induced by OVX in mice (FIG. 5D and E).

### EXAMPLE 2

### α-KG and NMN Synergistically Prevents High Fat Diet-Induced Obesity and Bone Loss

Mice were given a regular diet or high fat diet (HFD) for three months beginning at age of 8 weeks. The mice were about 25 grams at the start of the study. HFD-fed mice were feed with regular drinking water or drinking water containing α-KG and/or NMN to provide the following daily dose levels: 1) 100 mg/kg/day α-KG; 2) 100 mg/kg/day NMN; 3) 500 mg/kg/day α-KG; 4) 500 mg/kg/day NMN; and 5) combination 100 mg/kg/day α-KG+100 mg/kg/day NMN. Body weight, food and water consumption were monitored on a weekly basis for three months. Nuclear magnetic resonance scanner was used to assess lean mass and fat mass composition. Liver and adipose tissues were harvested for histology and histomorphometric analyses. Femur, tibia and mandible were subjected to micro-CT scanning and reconstruction. For marrow adipose tissue analyses, tibias were decalcified in 1:1 ratio of 2% osmium tetroxide and 5% potassium dichromate solution for 2 days and then micro-CT scanning was conducted. One-way ANOVA was performed to quantify differences among all the groups.

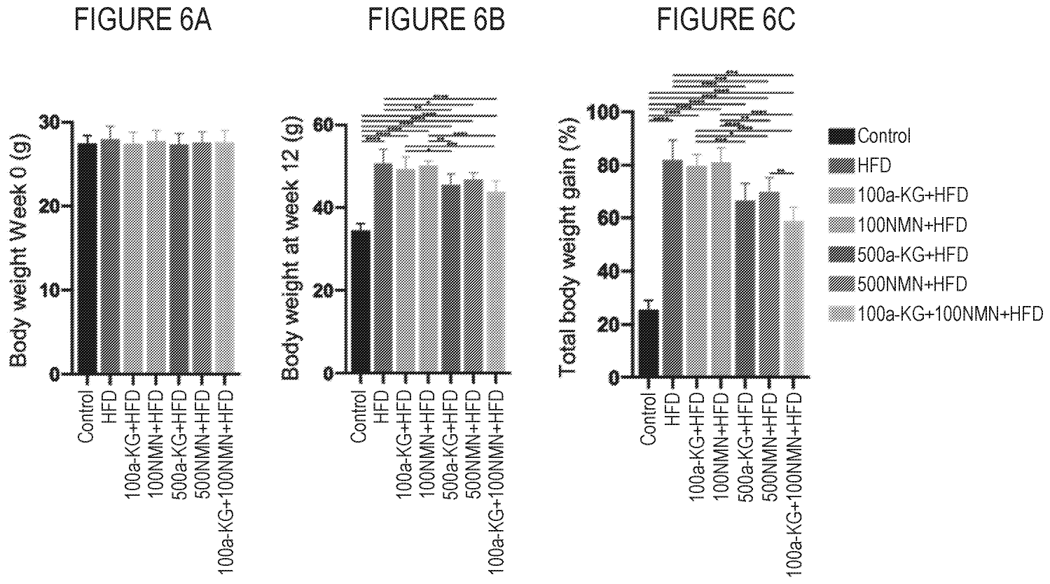

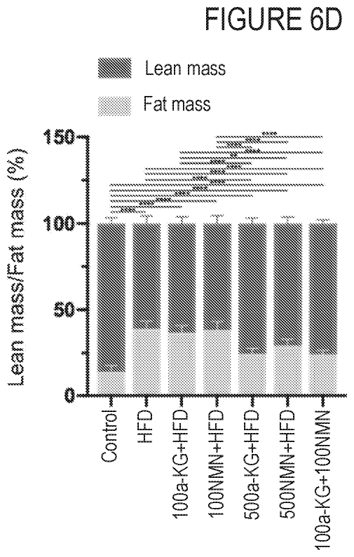

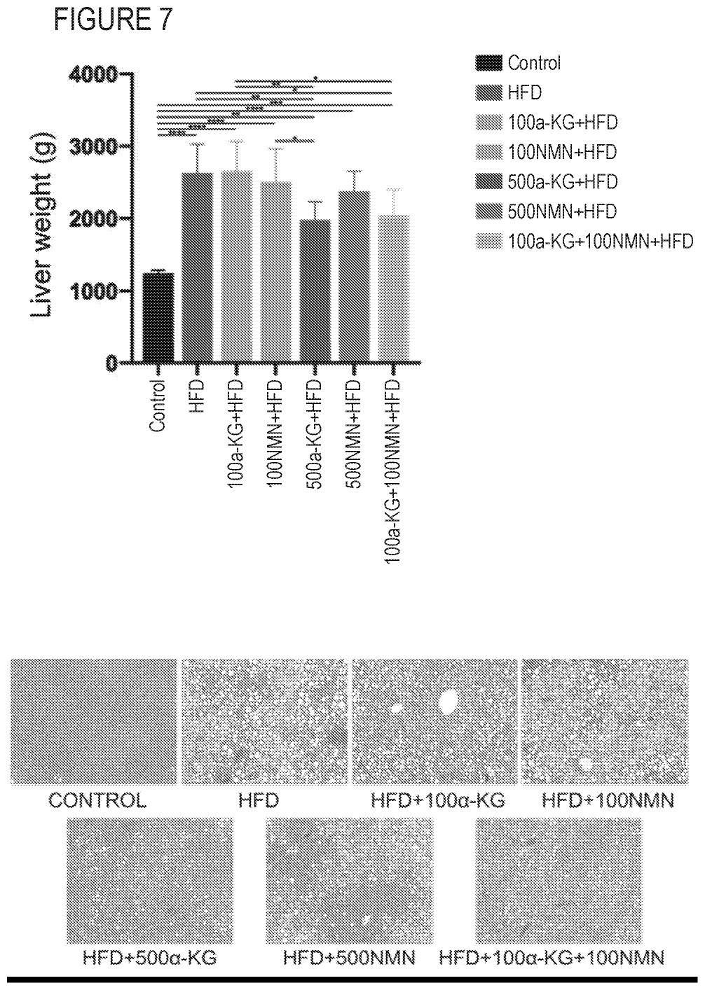

Mice fed with HFD gained significantly more weight compared with the control group. While the low concentration of α-KG or NMN alone did not significantly reduce weight gains induced by HFD, the combination of the low concentrations of α-KG and NMN significantly inhibited weight gain induced by HFD (FIG. 6A-C). Nuclear magnetic resonance analyses demonstrated that HFD significantly increased higher fat mass and lower lean mass compared with the control group. While 100 mg/kg/day α-KG or 100 mg/kg/day NMN could not affect fat mass, their combination significantly reduced higher fat mass induced by HFD (FIG. 6D). Similarly, the combination of α-KG+NMN also synergistically reduced liver weight and lipid accumulation (FIG. 7A and B).

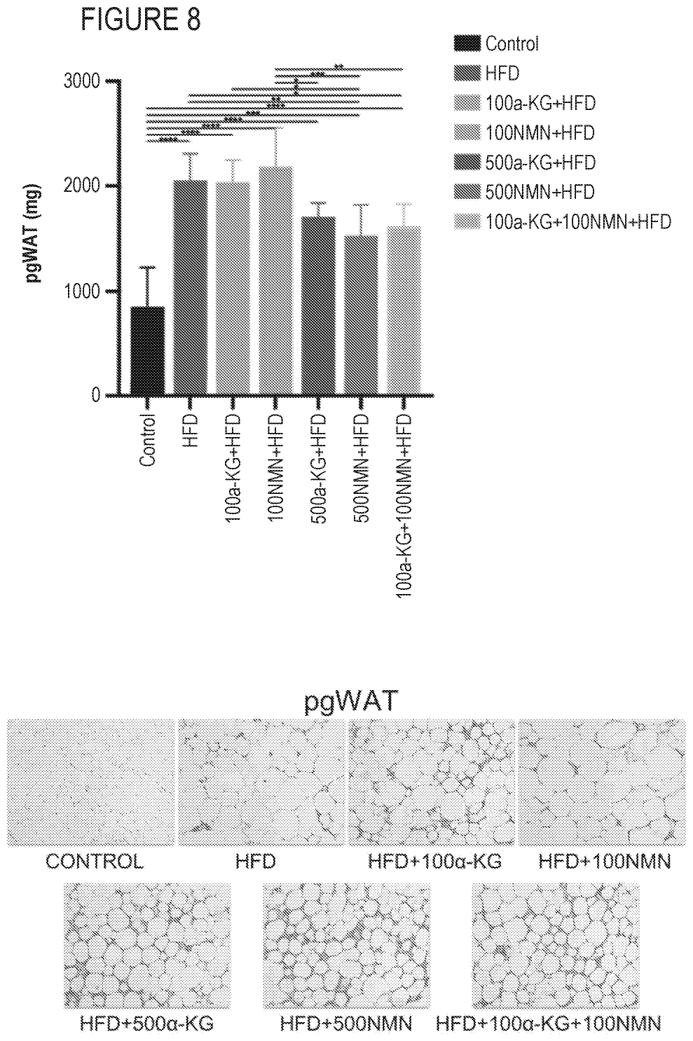

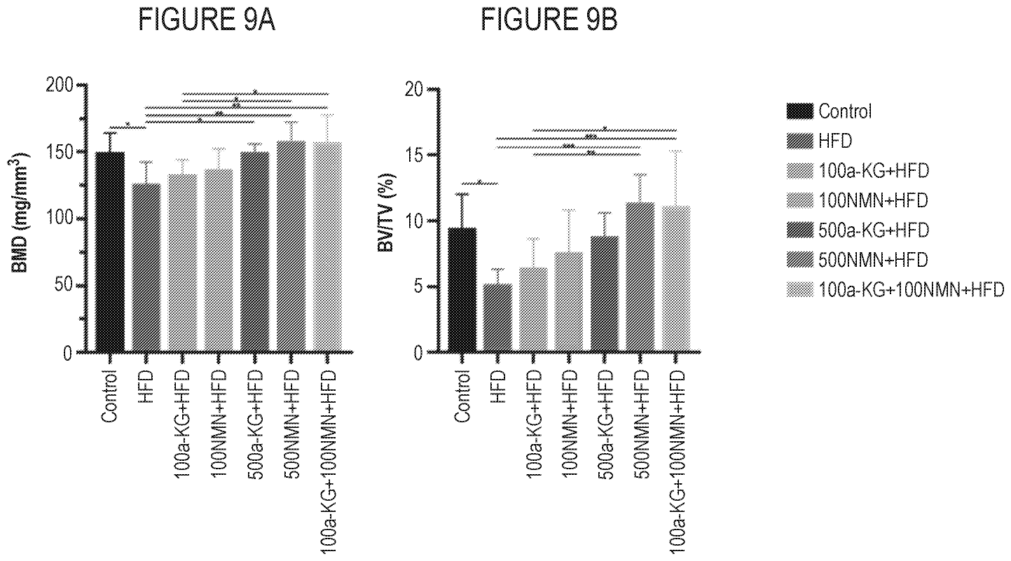

HFD significantly increased the weight and size of pgWAT compared with the control group. While the low concentration of α-KG or NMN did not significantly inhibit pgWAT weight gain induced by HFD, the combination of α-KG and NMN significantly inhibited them (FIG. 8A). Moreover, histological analysis also showed that the combination of α-KG and NMN significantly reduced the lipid droplets sizes compared with α-KG or NMN treatment alone (FIG. 8B). μCT analyses demonstrated that femur trabecular BMD and bone volume to trabecular volume ratio (BV/TV) in mice decreased significantly upon HFD. The combination of α-KG and NMN significantly prevented bone loss compared with α-KG or NMN treatment alone.

### EXAMPLE 3

### α-KG and NMN Synergistically Prevents Bone and Adipose Metabolism during Aging

Two separate batches of in vivo experiment were conducted to investigate the effect of α-KG and NMN on bone and adipose metabolism in aged C57BL/6J male mice. The sample size was 8 in total for each group, and the drinking water provided the following daily doses: 1) control; 2) 100 mg/kg/day α-KG; 3) 100 mg/kg/day NMN; 4) 500 mg/kg/day α-KG; 5) 500 mg/kg/day NMN; and 6) combination 100 mg/kg/day α-KG+100 mg/kg/day NMN). α-KG or NMN, or the combination, were delivered in drinking water starting from age of 18 months and continued for six months. Mice weighed 40 g on average at the start of the study. Body weight, food and water consumption were monitored on a weekly basis. Nuclear magnetic resonance scanner was used to assess lean mass and fat mass composition. Liver and adipose tissues were harvested for histology and histomorphometric analyses. Femur, tibia and mandible were subjected to μCT scanning and reconstruction. For marrow adipose tissue analyses, tibias were decalcified in 1:1 ratio of 2% osmium tetroxide and 5% potassium dichromate solution for 2 days and then micro-CT scanning was conducted. One-way ANOVA was performed to quantify differences among all the groups.

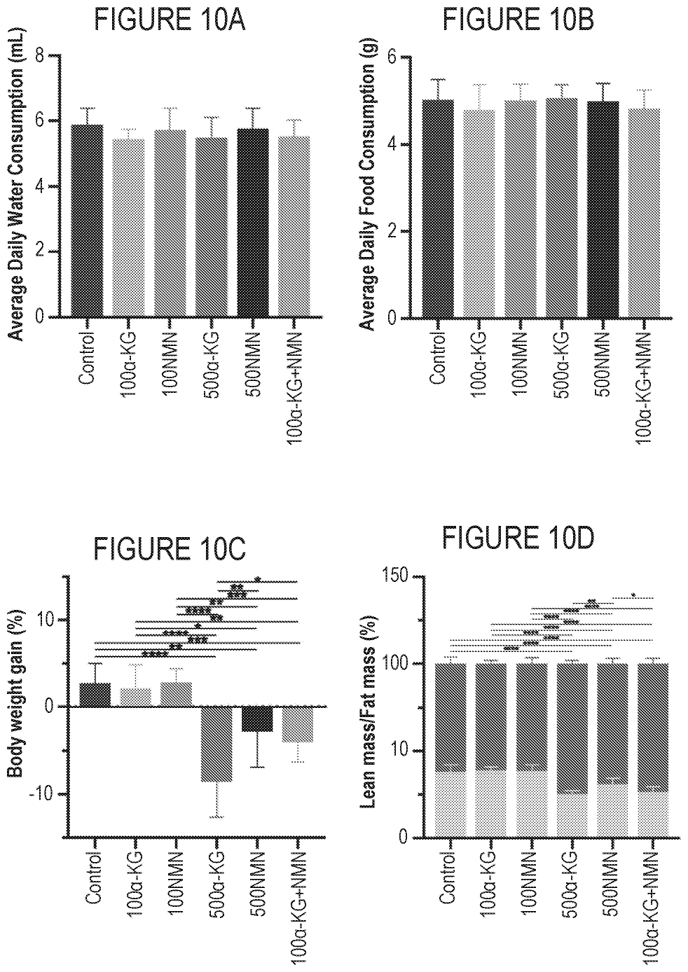

During the entire experiment, no significant differences of food and water intake were detected among all the groups (FIG. 10A-B). The body weight of control, 100 mg/kg/day α-KG and 100 mg/kg/day NMN groups increased at a similar rate after six months treatment. However, the body weights of 500 mg/kg/day α-KG, 500 mg/kg/day NMN and combination 100 mg/kg/day α-KG+100 mg/kg/day NMN groups decreased significantly after treatment (FIG. 10 C). Among those three groups whose body weight decreased, 500 mg/kg/day α-KG group presented significantly higher body weight drop than two other groups. No significant differences were observed between 500 mg/kg/day NMN and combination 100 mg/kg/day α-KG+100 mg/kg/day NMN groups. Nuclear magnetic resonance analyses demonstrated that the control, 100 mg/kg/day α-KG and 100 mg/kg/day NMN groups represented significantly higher fat mass and lower lean mass compared with 500 mg/kg/day α-KG, 500 mg/kg/day NMN and combination 100 mg/kg/day α-KG+100 mg/kg/day NMN groups (FIG. 10D). Among all the groups, 500 mg/kg/day α-KG groups presented the lowest fat mass and the highest lean mass.

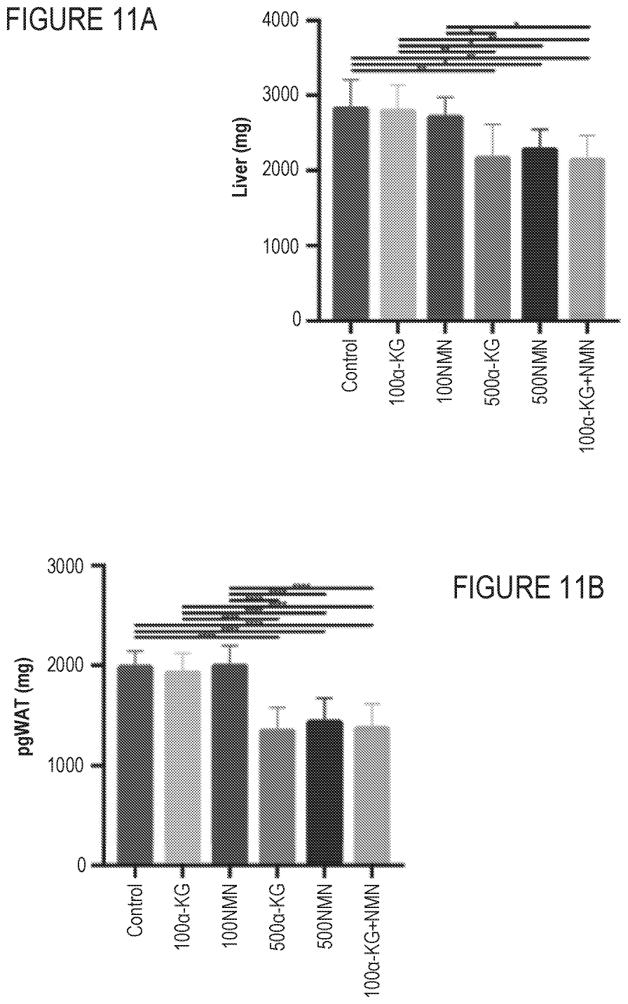

The liver plays a key role in lipid metabolism. The weight and size of liver were significantly higher in control, 100 mg/kg/day α-KG, and 100 mg/kg/day NMN groups compared with 500 mg/kg/day α-KG, 500 mg/kg/day NMN and the combination 100 mg/kg/day α-KG+100 mg/kg/day NMN groups (FIG. 11A).

There are two types of adipose tissues found in mammals, brown adipose tissue and white adipose tissue. In rodents, brown adipose tissue (BAT) is distributed throughout the fat pads with the main BAT depot in the interscapular region and is notable for its regulatory function in metabolism and adaptive thermogenesis. White adipose tissue (WAT) is stored in both visceral and subcutaneous depots and is served as a primary energy reservoir. A frequently analyzed WAT is peri-gonadal WAT (pgWAT). The weight of pgWAT was significantly higher in control, 100 mg/kg/day α-KG and 100 mg/kg/day NMN groups, compared with 500 mg/kg/day α-KG, 500 mg/kg/day NMN and combination 100 mg/kg/day α-KG+100 mg/kg/day NMN groups (FIG. 11B).

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

What is claimed is:

1. A method for treating skeletal aging, osteoporosis or obesity in a subject comprising administering to the subject therapeutically effective amounts of alpha-ketoglutarate (α-KG) and nicotinamide mononucleotide (NMN).

2. The method of claim 1 wherein the therapeutically effective amounts of alpha-ketoglutarate (α-KG) and nicotinamide mononucleotide (NMN) are synergistic in treating skeletal aging, osteoporosis or obesity.

3. The method of claim 1 wherein the administering promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof.

4. The method of claim 1 wherein the administration decreases body weight during aging.

5. The method of claim 1 wherein the α-KG and NMN are administered in the same dosage form.

6. The method of claim 1 wherein the α-KG and NMN are administered in separate dosage forms.

7. The method of claim 1 wherein the α-KG and NMN are each administered daily.

8. The method of claim 1 wherein the α-KG and the NMN are each administered orally.

9. The method of claim 1 wherein the α-KG and the NMN are administered chronically.

10. The method of claim 1 wherein the subject is overweight, obese, has a high BMI, has type 1 diabetes mellitus, has undergone radiation therapy or has undergone chemotherapy.

11. The method of claim 1 wherein the subject has a Western diet.

12. The method of claim 1 wherein the dose of α-KG is about 10-1000 mg/day and the dose of NMN is about 10-1000 mg/day.

13. The method of claim 11 wherein the dose of α-KG is about 10-500 mg/day and the dose of NMN is about 10-500 mg/day.

14. The method of claim 11 wherein the dose of α-KG is about 100-500 mg/day and the dose of NMN is about 100-500 mg/day.

15. The method of clam 1 wherein the NMN is nicotinamide riboside.

16. A method for promoting osteogenic differentiation, increasing in trabecular bone, inhibiting adipogenic differentiation of bone marrow mesenchymal cells, inhibiting adipogenic differentiation of bone marrow stromal cells, increasing bone mass, reducing bone marrow adiposity, preventing bone loss, increasing the ratio of osteogenesis to adipogenesis in bone marrow mesenchymal cells, or any combination thereof, in a subject comprising administering to the subject a synergistic combination of α-KG and NMN for a duration sufficient to be effective.

17. The method of claim 16 wherein the α-KG and NMN are administered in the same dosage form.

18. The method of claim 16 wherein the α-KG and NMN are administered in separate dosage forms.

19. The method of claim 16 wherein the α-KG and NMN are each administered daily.

20. The method of claim 16 wherein the α-KG and the NMN are each administered orally.

21. The method of claim 16 wherein the α-KG and the NMN are administered chronically.

22. The method of claim 16 wherein the dose of α-KG is about 10-1000 mg/day and the dose of NMN is about 10-1000 mg/day.

23. The method of claim 16 wherein the dose of α-KG is about 10-500 mg/day and the dose of NMN is about 10-500 mg/day.

24. The method of claim 16 wherein the dose of α-KG is about 100-500 mg/day and the dose of NMN is about 100-500 mg/day.

25. The method of clam 16 wherein the NMN is nicotinamide riboside.

26. A composition comprising therapeutically effective amounts of alpha-ketoglutarate (α-KG) and nicotinamide mononucleotide (NMN) for treating skeletal aging, osteoporosis or obesity in a subject.

27. The composition of claim 26 wherein the therapeutically effective amounts of alpha-ketoglutarate (α-KG) and nicotinamide mononucleotide (NMN) are synergistic in treating skeletal aging, osteoporosis or obesity.

28. The composition of claim 26 wherein the treating promotes osteogenic differentiation, increase in trabecular bone, inhibits adipogenic differentiation of bone marrow mesenchymal cells, inhibits adipogenic differentiation of bone marrow stromal cells, increases bone mass, reduces bone marrow adiposity, prevents bone loss, increases the ratio of osteogenesis to adipogenesis in bone marrow mesenchymal cells, or any combination thereof.

29. The composition of claim 26 wherein the treating decreases body weight during aging.

30. The composition of claim 26 provided for daily administration.

31. The composition of claim 26 provided for oral administration.

32. The composition of claim 26 wherein the dose of α-KG is 10-1000 mg and the dose of NMN is 10-1000 mg.

33. The composition of claim 26 wherein the dose of α-KG is about 10-500 mg/day and the dose of NMN is about 10-500 mg/day.

34. The composition of claim 26 wherein the dose of α-KG is about 100-500 mg/day and the dose of NMN is about 100-500 mg/day.

35. The composition of clam 26 wherein the NMN is nicotinamide riboside.

36. The composition of claim 26 wherein the combination is a synergistic combination.
